(19) United States
(12) Patent Application Publication
(10) Pub. No.: US 2002/0112721 A1
(43) Pub. Date: Aug. 22, 2002

(54) Kayak breathing system and method therefor

(76) Inventor: Michael R. Beier, Schaumburg, IL (US)

Correspondence Address: DILLIS V. ALLEN, 1080 Nerge Road, Suite 205, Elk Grove Village, IL, 60007, US

(21) Appl. No.: 09/790,156

(22) Filed: Feb. 21, 2001

(51) Int. Cl. A62B 7/00; A62B 9/00; A61M 15/00; A61M 16/00; A62B 18/00

(52) U.S. Cl. 128/200.24

(57) ABSTRACT

A method and apparatus for breathing in a Kayak with a mouth piece and a flexible tube including positioning the mouth piece adjacent the Kayaker's head, positioning the tube so that it extends downwardly into the Kayak interior and freely communicates with hull interior air, inserting the mouth piece when necessary into the mouth and breathing Kayak interior air.

### BACKGROUND OF THE INVENTION

Kayaking has become considerably more popular over the last decade and is in fact one of the world's fastest growing sports in terms of the increase in the number of Kayaks sold. It is estimated that there are 5 million Kayakers in the United States alone. This burgeoning popularity is due in part to the adventurous nature of Kayaking, particularly in rough and rapid moving water, but also is due in part to the technological development of the Kayak itself, including molded seating systems, improved foot rests, and improved interior floatation arrangements.

The Kayak system usually includes a skirt that is sealed to the Kayak deck opening and wraps around the torso and vest of the Kayaker to prevent the ingress of water into the interior of the Kayak.

This system is particularly useful when the Kayak capsizes and the Kayaker is “turtled” under the water. In this mode, the skirt prevents water from entering the hull and enables the Kayaker to execute a maneuver called an “eskimo roll” whereby the Kayaker rights the Kayak without wet exiting the Kayak.

However, because of inexperience, fear, and an inability to breathe underwater, many Kayakers, even ones with substantial experience, must wet exit the boat when capsized and drag it to shore, empty it, and reboard. This is a laborious and time-consuming task. There are many Kayak classes where the students attempt to learn the “eskimo roll” but are hindered by the inability to breathe underwater.

Thus, there is a need in Kayaking to provide a breathing system for the Kayaker, particularly when the Kayak capsizes.

In the Dusenbery, U.S. Pat. No. 5,887,585, a Water Rafting Canoeing and Kayaking Safety Vest is disclosed having front rear panels that incorporate sections of a floatation material, wherein the floatation material receives and stores miniature scuba-compressed air tanks.

This system has not proved commercially viable because the vest is too bulky and interferes with the Kayak skirt. Moreover, it is uncomfortable and heavy making oar manipulation by the Kayaker more difficult.

A somewhat different system is shown in the Schoettle, U.S. Pat. No. 5,671,694 , wherein an Air System for a Kayak is provided that includes a pair of similar air bags having an input valve for filling the bags and an outlet orifice for providing air to the Kayaker.

The principal disadvantage in this system is that the air bags are small and are not pressurized, and hence, do not provide the Kayaker with sufficient breathing time to execute the “eskimo roll”. Furthermore, in this system the Kayaker exhales into the air bags, filling them with carbon dioxide and thus decreasing breathing quality. Also, the Schoettle system provides no means for purging the mouth piece of air.

It is a primary object of the present invention to ameliorate the problems noted above in Kayak air breathing systems.

### SUMMARY OF THE PRESENT INVENTION

In accordance with the present invention, a Kayak breathing system and method are provided particularly designed for breathing in difficult situations such as when the Kayak is turtled and the Kayaker's head submerged.

Toward these ends a mouth piece is held near the Kayaker's mouth with a vest mounted holding device, a tube having a flexible portion is connected to the mouth piece and runs through the vest, and held by the vest downwardly (when upright) into the Kayak interior. The open end of the tube freely connects with the hull interior air and a floatation device on the end of the tube keeps the tube exposed to air and not water when the interior partly fills with water.

A first check valve in the tube permits the expulsion of water from the tube by exhaling. A second check valve in the tube prevents water in the tube from traveling to the Kayak interior.

### BRIEF DESCRIPTION OF THE DRAWINGS

FIG. 1 is a longitudinal section of a Kayak showing the present Kayak breathing system;

FIG. 2 is a cross section approximately midway through the Kayak with the Kayak inverted or capsized and the Kayaker submerged;

FIG. 3 is an exploded view of the Kayak breathing system according to the present invention;

FIG. 4 is an assembled view of the Kayak breathing system illustrated in FIG. 3;

FIG. 5 is an end view of one of the check valves illustrated in FIG. 3;

FIG. 6 is a side view of one of the check valves illustrated in FIG. 3;

FIG. 7 is an end view of the check valve sub-assembly;

FIG. 8 is a side view of the check valve sub-assembly, and;

FIG. 9 is a longitudinal section through the check valve illustrated in FIG. 6.

### DETAILED DESCRIPTION OF THE PREFERRED EMBODIMENT

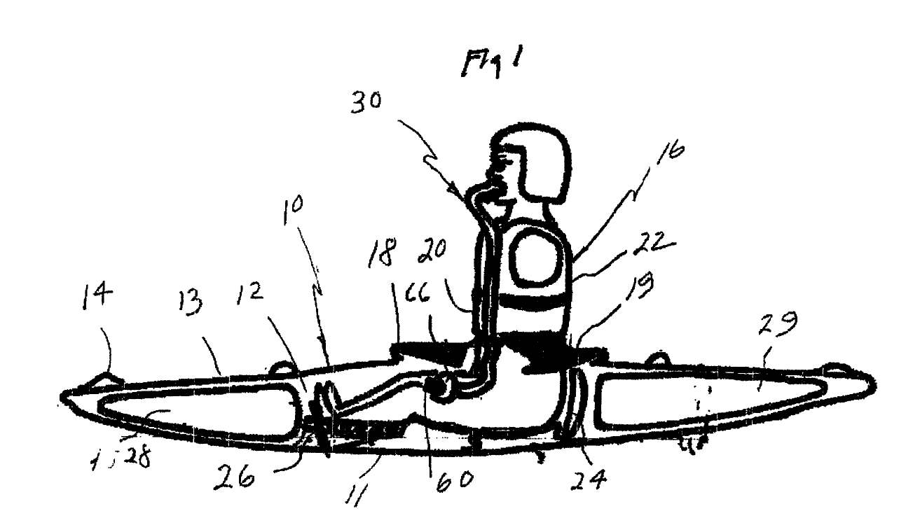

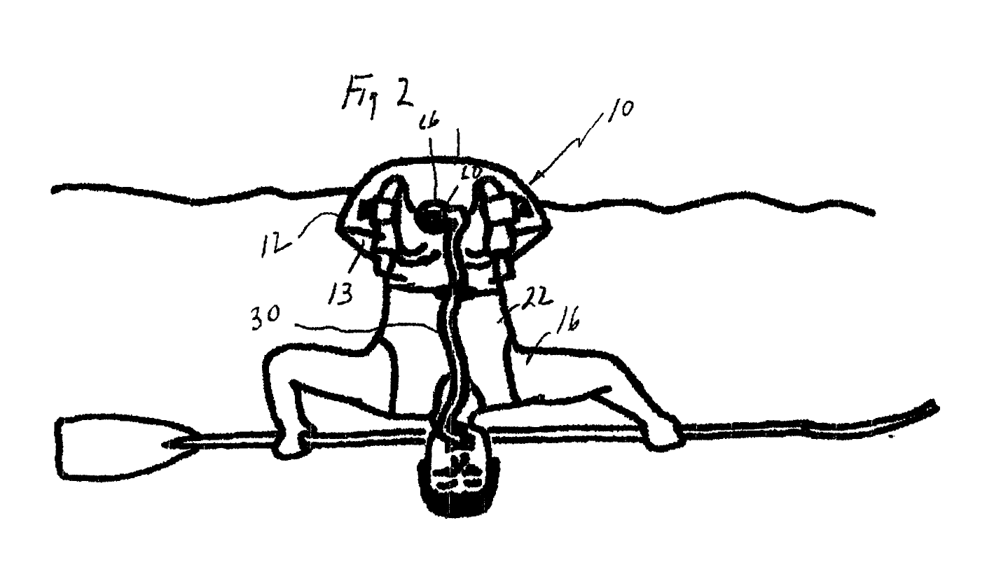

Referring to the drawings and particularly FIGS. 1 and 2, it should be understood that the Kayak 10 illustrated in both of these views is schematically illustrated in longitudinal section in FIG. 1, and an approximate mid-ship cross section is shown in FIG. 2, where the Kayak is “turtled” or inverted when capsized.

Kayak 10 is seen to include a hull portion 11, a gunnel 12, and a top deck 13. The top deck has a plurality of hand loops 14 thereon for manually handling the Kayak. A Kayaker 16 is also illustrated in FIGS. 1 and 2, although without arms in FIG. 1 simply for clarity. The Kayaker is positioned in the Kayak in the rowing position within a central generally oval opening 18 therein. Opening 18 is covered by a skirt, which has a flared portion 19 sealed to the opening 18 and an integral tube portion 20 that laces or is otherwise tied around the Kayaker's torso partly covering vest 22. The Kayaker sits on a molded plastic seat 24 having a back rest of a seat portion, and rests his feet on an adjustable foot rack 13. A bow floatation device 28 is positioned within the hull in the bow and rear floatation device 29 is positioned in the stern behind the Kayaker 16.

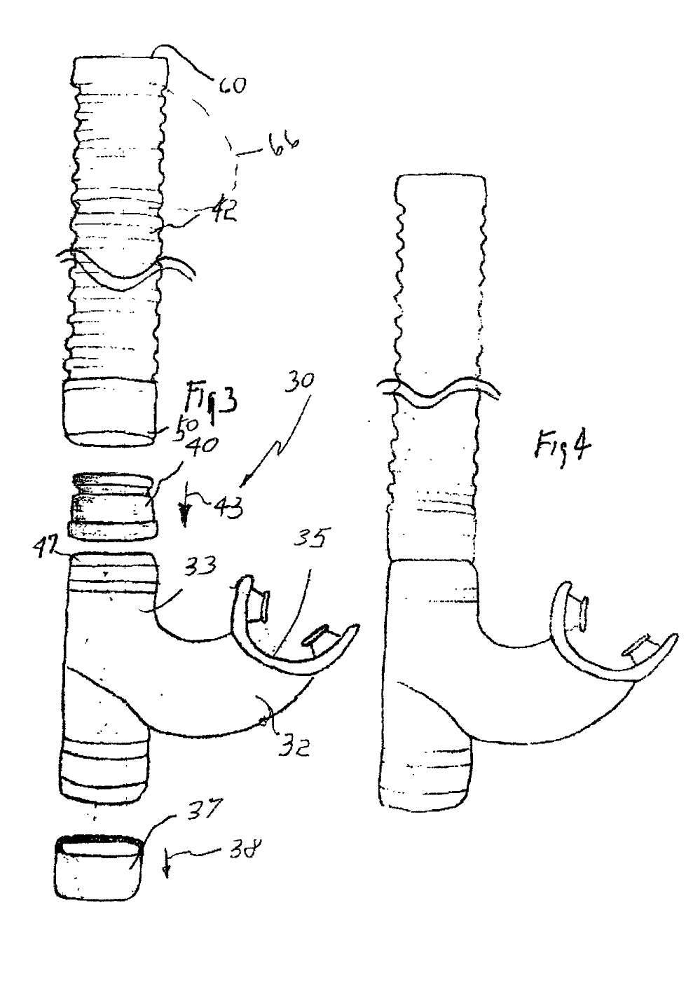

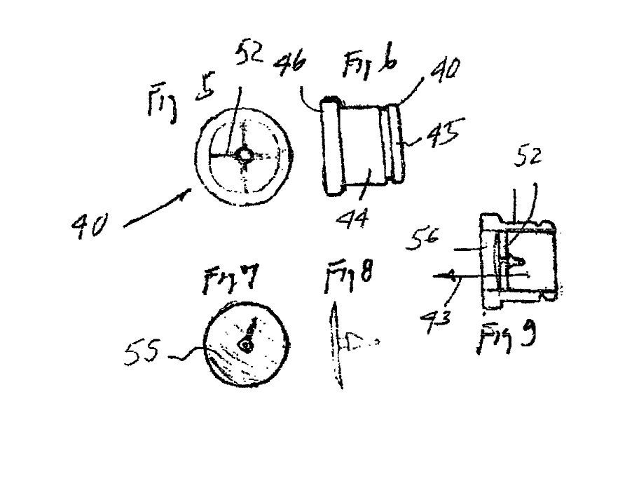

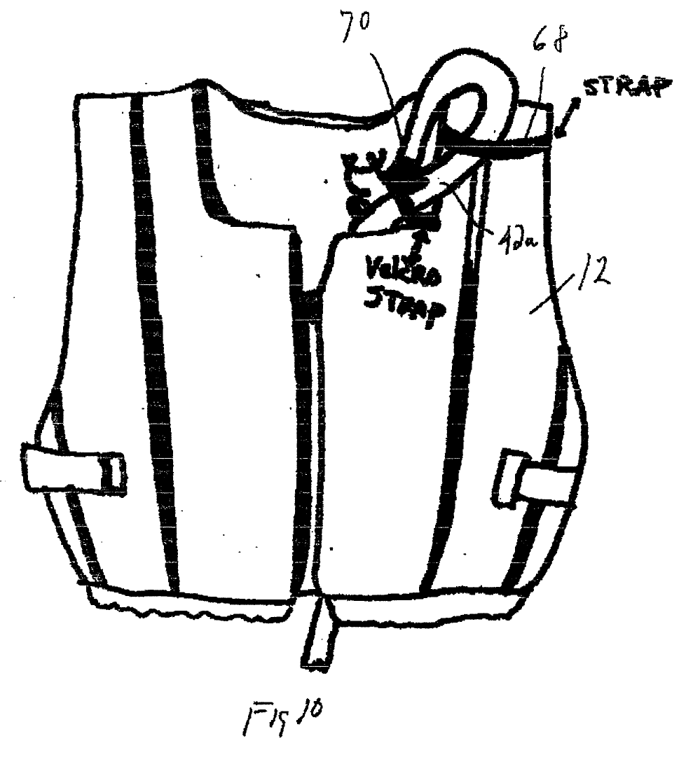

A breathing system 30, according to the present invention, is shown generally in schematic form in FIGS. 1 and 2, and as seen in FIG. 4, includes a rigid mouth piece assembly 32 having an integral short straight tubular section 33 communicating with mouth piece opening 35. A first check valve 37 permits exhaled air to purge water in tube 33 and mouth piece assembly 32 and opens only in the direction of arrow 38. Also when breathing, check valve 37 discharges exhaled air into the surrounding water. A second check valve assembly 40 prevents water from entering flexible tube section 42 when the mouth piece is not in the Kayaker's mouth and opens only in the direction of arrow 43. Both of the check valves 37 and 40 are similar in construction and the check valve 40 is illustrated in detail in FIGS. 5 to 9. As seen in these views, check valve 40 includes a stepped annular outer section 44 having a male end 45 and a female end 46 adapted to mate respectively with tubular portion 50 on tube 42 and tubular portion 47 on tube 33. The outer section 44 has a seat 52 defined by four struts separated by a screen section against which a movable check valve 55 seats as shown in the cross section of FIG. 9. When a vacuum is applied to valve side 56, valve 55 moves away from seat 52 permitting air to flow into the mouth piece 35. However, when water is applied to side 56, seat 53 closes and prevents the entry of water into the canoe hull through flexible tube 42.

Instead of check valve 40, a manually operated On/Off valve can be provided at the location of check valve 40. In some situations this On/Off valve can provide easier breathing than the check valve 40.

While not shown clearly in FIGS. 1 and 2, the flexible tube 42 threads inside the vest 22 and is held in position by the vest and it has an open end 60 that freely communicates with the interior of the Kayak so that the Kayaker, when breathing through the mouth piece 32, breathes atmospheric air within the Kayak hull. The check valve 40 prevents exhaled air and carbon dioxide from entering the hull and it passes freely out the check valve 37 into the water when in the turtled position illustrated in FIG. 2. A styrofoam sphere 66, or other types of floatation devices, is bonded to the end 60 of the tube 42 and prevents the open end of the tube 60 from becoming submerged in the event there is water in the Kayak when in the inverted position illustrated in FIG. 2. The vest 22 is shown more clearly in FIG. 10 and is seen to include a first “Velcro” type strap 68 for holding the mouth piece 32 around the shoulder strap portion of the vest 12 near the Kayaker's face so that it can be easily inserted into the mouth. A second Velcro strap 70 encompasses the mouth piece and an upper portion 42a of tube 42 to keep the flexible tube 42 in a bent position in the event that the check valve 40 is desired to be eliminated. That is, the bend in the tube 42 in FIG. 10 prevents water from entering the Kayak through the tube 42, and thus, is an alternative to the check valve 40. It should be understood, however, that the check valve 40 has a dual purpose of preventing the entry of water into the Kayak and also preventing the entry of exhaled carbon dioxide into the Kayak.

1. A breathing system for Kayakers, comprising:
a mouth piece, a holder for the mouth piece, and a tube having a flexible portion extending from the mouthpiece to a position inside the Kayak, said tube having an open end communicating with Kayak interior air.

2. A breathing system for Kayakers, comprising:
a vest, a mouthpiece, means on the vest holding the mouth piece adjacent the Kayaker's head, a tube having a flexible portion extending from the mouth piece downwardly into the Kayak interior, means on the vest holding the flexible tube in position, said tube having an open end communicating freely with the Kayak interior air.

3. A breathing system for Kayakers as defined in claim 2, including a first valve in the tube permitting exhaled air to escape from the tube without flowing into the Kayak interior.

4. A breathing system for Kayakers as defined in claim 3, wherein the first valve is a one-way check valve.

5. A breathing system for Kayakers as defined in claim 2, including a second valve in the tube for preventing water flow through the mouth piece into the Kayak interior.

6. A breathing system for Kayakers as defined in claim 5, wherein the second valve is a one-way check valve.

7. A breathing system for Kayakers as defined in claim 2, wherein the means on the vest to hold the mouth piece adjacent the Kayaker's head includes the interior surfaces of the vest itself.

8. A breathing system for Kayakers, comprising:
a vest, a mouthpiece, means on the vest holding the mouth piece adjacent the Kayaker's head, a tube having a flexible portion extending from the mouth piece downwardly into the Kayak interior, means on the vest holding the flexible tube in position, said tube having an open end communicating freely with the Kayak interior air, a first valve in the tube permitting exhaled air to escape from the tube without flowing into the Kayak interior, a second valve in the tube for preventing water flow through the mouth piece into the Kayak interior, and said means on the vest to hold the mouth piece adjacent the Kayaker's head also including hook and eye strips.

9. A method of breathing in a Kayak using a mouth piece and a flexible tube connected thereto, including the steps of positioning the mouth piece adjacent the Kayaker's head, positioning the flexible tube so it extends into the Kayak's interior, exposing the open end of the tube to interior air in the Kayak, and when desired or necessary, inserting the mouth piece into the Kayaker's mouth and breathing Kayak interior air.
